(19) United States
(12) Patent Application Publication
(10) Pub. No.: US 2024/0307693 A1
(43) Pub. Date: Sep. 19, 2024

(54) NEUROANATOMY-BASED COMPARATIVE SEARCH TO OPTIMIZE DEEP BRAIN STIMULATION

(71) Applicant: Boston Scientific Neuromodulation Corporation, Valencia, CA (US)

(72) Inventors: Mahsa Malekmohammadi, Sherman Oaks, CA (US); Richard Mustakos, Simi Valley, CA (US); Stephen Carcieri, Los Angeles, CA (US)

(21) Appl. No.: 18/598,460

(22) Filed: Mar. 7, 2024

Related U.S. Application Data
(60) Provisional application No. 63/451,705, filed on Mar. 13, 2023.

(51) Int. Cl. A61N 1/36; A61N 1/05; G16H 20/40

(52) U.S. Cl. CPC A61N 1/36175; A61N 1/0534; G16H 20/40

(57) ABSTRACT

This document discusses neurostimulation devices, systems, and methods. A neurostimulation system receives a physiological target region for the neurostimulation and one or more optimization criteria for the neurostimulation of the target region. The neurostimulation system determines a likelihood of finding a stimulation configuration solution using a first optimization algorithm according to the physiological target region, determines a likelihood that the stimulation configuration solution is a better solution than what would be found by a second optimization algorithm and selects the first optimization algorithm or the second optimization algorithm according to the determined likelihood. The neurostimulation system recurrently changes stimulation parameters according to the selected optimization algorithm to determine the stimulation configuration solution based on the one or more optimization criteria and presenting the stimulation configuration solution to a user.

### CLAIM OF PRIORITY

This application claims the benefit of U.S. Provisional Application No. 63/451,705 filed on Mar. 13, 2023, which is hereby incorporated by reference in its entirety.

### TECHNICAL FIELD

This document relates generally to medical devices, and more particularly, to systems, devices, and methods for determining and setting of stimulation parameters for programming an electrical neurostimulation system.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on an intricate balance of stimulation location coupled with the programmed stimulation waveform. However, the number of programmable options can create an extensive parameter search space for the physician or clinician. Finding the optimal neurostimulation parameters may take a lot of time in the clinic for both the clinic staff and the patient.

### SUMMARY

In DBS, electrical neurostimulation therapy is delivered to implantable electrodes located at certain neurostimulation targets in the brain to treat neurological or neurophysiological disorders. Device-based neurostimulation can include techniques to reduce the parameter search space for the physician when customizing neurostimulation parameters to a particular patient.

Example 1 includes subject matter (such as a machine-implemented method of controlling operation of a neurostimulation system) comprising receiving, by the neurostimulation system, a physiological target region for the neurostimulation; receiving one or more optimization criteria for the neurostimulation of the target region; determining a likelihood of finding a stimulation configuration solution using a first optimization algorithm according to the physiological target region and the one or more optimization criteria; determining a likelihood that the stimulation configuration solution found by the first optimization algorithm is a better stimulation configuration solution than a stimulation configuration solution that would be found by a second optimization algorithm; selecting the first optimization algorithm or a second optimization algorithm according to the determined likelihoods; and recurrently changing stimulation parameters according to the selected optimization algorithm to determine the stimulation configuration solution based on the one or more optimization criteria and presenting the stimulation configuration solution to a user.

In Example 2, the subject matter of Example 1 optionally includes finding a solution electrode configuration, the electrode configuration specifying a selection of one or more electrodes from the plurality of electrodes and a fractionalization of electrical current flowing through the selected one or more electrodes. The first optimization algorithm optionally uses a gradient descent approach to find the solution electrode configuration and the second optimization algorithm tests every available electrode configuration of the neurostimulation system to find the stimulation configuration solution.

In Example 3, the subject matter of one or both of Examples 1 and 2 optionally includes receiving, by the neurostimulation system, one or more avoidance regions for the neurostimulation; determining a weighted summation including a stimulated target volume of the target region, a stimulated avoidance volume of the one or more avoidance regions, and a total stimulated volume; and determining the stimulation configuration solution according to the weighted summation.

In Example 4, the subject matter of one or any combination of Examples 1-3 optionally includes searching a database of stored stimulation setting results of a patient population, the stored stimulation setting results determined using the first optimization algorithm; and determining the likelihoods according to matching the received target region to target regions for the stored stimulation setting results of the database.

In Example 5, the subject matter of Example 4 optionally includes determining the likelihood of finding the stimulation configuration solution according to matching one or more electrode configurations of the neurostimulation system to electrode configurations for the stored stimulation setting results of the database.

In Example 6, the subject matter of one or both of Examples 4 and 5 optionally includes determining, when the first optimization algorithm is selected, test stimulation configurations using stimulation parameters of matched stored stimulation setting results of the database.

In Example 7, the subject matter of one or any combination of Examples 1-6 optionally includes optimization criteria including a ratio including the total tissue volume activated by the neurostimulation and the tissue volume outside of the target region activated by the neurostimulation.

In Example 8, the subject matter of one or any combination of Examples 1-7 optionally includes optimization criteria including optimization of one or both of stimulation amplitude and total charge delivered to the tissue of the patient.

In Example 9, the subject matter of one or any combination of Examples 1-8 optionally includes receiving, by the neurostimulation system, one or more avoidance regions for the neurostimulation; and identifying a simulation configuration solution using a decision criterion that includes a value of a metric that includes a weighted summation of a stimulated target volume of the target region, a stimulated avoidance volume of the one or more avoidance regions, and a total stimulated volume of the tissue of the patient.

Example 10 includes subject matter (such as a system for delivering neurostimulation to tissue of a patient using multiple electrodes) or can optionally be combined with one or any combination of Examples 1-9 to include such subject matter, comprising a stimulation control circuit to deliver the neurostimulation according to a specified stimulation configuration, the stimulation configuration including multiple stimulation parameters; a port to receive a designation of a target region for the neurostimulation; a user interface configured to receive one or more optimization criteria for the neurostimulation of the target region; and a programming control circuit. The programming control circuit is configured to specify multiple stimulation configurations; perform multiple optimization algorithms, each algorithm to determine a stimulation configuration solution from the multiple stimulation configurations; determine a likelihood of finding a stimulation configuration solution using a first optimization algorithm according to the target region and the one or more optimization criteria; determine a likelihood that the stimulation configuration solution found by the first optimization algorithm is a better stimulation configuration solution than a stimulation configuration solution that would be found by a second optimization algorithm; select the first optimization algorithm or another optimization algorithm according to the determined likelihoods; and recurrently change stimulation parameters according to the selected optimization algorithm to determine the stimulation configuration solution based on the one or more optimization criteria and present the stimulation configuration solution using the user interface.

In Example 11, the subject matter of Example 10 optionally includes a programming control circuit configured to specify electrode configurations that include a selection of one or more electrodes of the multiple electrodes; identify, when performing the first optimization algorithm, an initial set of stimulation settings, find an approximate stimulation configuration solution using the initial set of stimulation settings, and identify a next set of stimulation settings based on the approximate stimulation configuration solution; and when performing the other optimization algorithm, test every available electrode configuration of the multiple electrodes when finding the stimulation configuration solution.

In Example 12, the subject matter of one or both of Examples 10 and 11 optionally includes a programming control circuit configured to receive one or more avoidance regions for the neurostimulation; determine the stimulation configuration solution using a weighted summation including a stimulated target volume of the target region, a stimulated avoidance volume of the one or more avoidance regions, and a total stimulated volume; and determine the stimulation configuration solution according to the weighted summation.

In Example 13, the subject matter of one or any combination of Examples 10-12 optionally includes a storage device to store a database of stimulation setting results determined for a patient population using the first optimization algorithm and a programming control circuit configured to search the database to identify stored stimulation setting results for the received target region; and determine the likelihood of finding the stimulation configuration solution according to the identified stored stimulation setting results.

In Example 14, the subject matter of Example 13 optionally includes a programming control circuit configured to select one or more electrode configurations of the multiple electrodes to activate the target region; and determine the likelihood of finding the stimulation configuration solution and determine the likelihood that the stimulation configuration solution is the better stimulation configuration according to matching the one or more electrode configurations of the neurostimulation system to electrode configurations for the stored stimulation setting results of the database.

In Example 15, the subject matter of one or both of Examples 13 and 14 optionally includes a programming control circuit configured to recurrently change stimulation parameters to stimulation settings of matched stored stimulation setting results of the database when using the first optimization algorithm to determine the stimulation configuration solution.

In Example 16, the subject matter of one or any combination of Examples 13-15 optionally includes a programming control circuit configured to search the database to identify stored stimulation setting results that include at least one optimization criterion of the received one or more optimization criteria; and determine the likelihood of finding the stimulation configuration solution according to the stored stimulation setting results identified according to the at least one optimization criterion.

In Example 17, the subject matter of Example 16 optionally includes an optimization criterion including a weighting of activation of tissue outside the target region.

Example 18 can include subject matter (or can optionally by combined with one or any combination of Examples 10-17 to include such subject matter) such as a computer readable storage medium including instructions that when performed by a programming control circuit of a neurostimulation device, cause the neurostimulation device to perform actions including receiving, by the neurostimulation device, a physiological target region of a subject for neurostimulation; receiving one or more optimization criteria for the neurostimulation of the target region; determining a likelihood of finding a stimulation configuration solution using a first optimization algorithm according to the physiological target region and the one or more optimization criteria; determining a likelihood that the stimulation configuration solution found by the first optimization algorithm is a better stimulation configuration solution than a stimulation configuration that would be found by a second optimization algorithm; selecting the first optimization algorithm or a second optimization algorithm according to the determined likelihood; and recurrently changing stimulation parameters according to the selected optimization algorithm to determine the stimulation configuration solution based on the one or more optimization criteria and presenting the stimulation configuration solution to a user.

In Example 19, the subject matter of Example 18 optionally includes a computer readable storage including instructions that cause the neurostimulation device to search a database of stored stimulation setting results of a patient population stored in a storage device, the stored stimulation setting results determined using the first optimization algorithm; and determine the likelihood of finding the stimulation configuration solution according to matching the received target region to target regions for the stored stimulation setting results of the database.

In Example 20, the subject matter of one or both of Examples 18 and 19 optionally includes a computer readable storage including instructions that cause the neurostimulation device to identify, when performing the first optimization algorithm, an initial electrode configuration, wherein an electrode configuration specifies a selection of one or more electrodes from the plurality of electrodes and a fractionalization of electrical current flowing through the selected one or more electrodes, find an approximate electrode configuration solution using the initial electrode configuration, and identify a next electrode configuration based on the approximate electrode configuration solution; select a solution electrode configuration from the candidate electrode configurations; and test, when performing the second optimization algorithm, every electrode configuration available to the neurostimulation system to determine the solution electrode configuration.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of an electrical neurostimulation system.

FIG. 2 is an illustration of portions of another example of an electrical neurostimulation system.

FIG. 3 is an illustration of an example of an implantable pulse generator (IPG).

FIG. 4 illustrates an embodiment of an IPG and an implantable lead system.

FIG. 5 is an illustration of an example of a programming device of a neurostimulation system.

FIG. 6 is an illustration of an implanted DBS stimulation lead.

FIGS. 7A-7G are illustrations of an example of electrodes of a stimulation lead.

FIGS. 8A-8B illustrate an example of a presentation of a stimulation configuration using a user interface of a neurostimulation system.

FIG. 9 is a flow diagram of a method to operate a neurostimulation system.

FIGS. 10A-18E are graphs of results of examples of finding a stimulation configuration solution using different optimization algorithms.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical, and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS therapy.

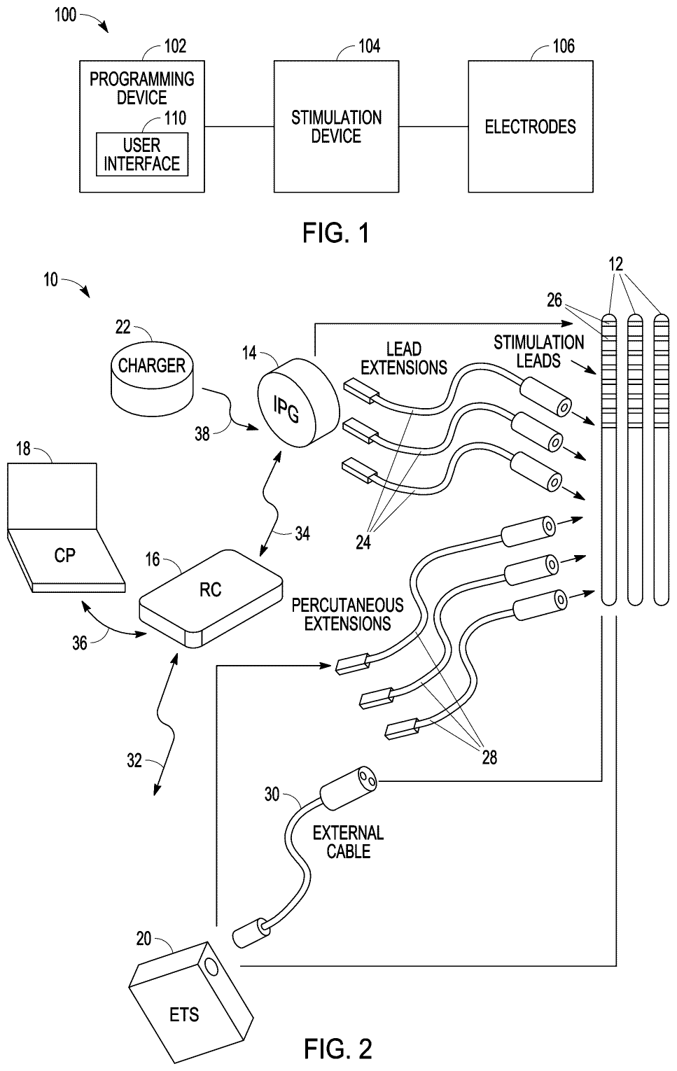

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. Such DBS configuration includes various features that may simplify the task of the user in programming the stimulation device 104 for delivering DBS to the patient, such as the features discussed in this document.

FIG. 2 is an illustration of portions of an embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

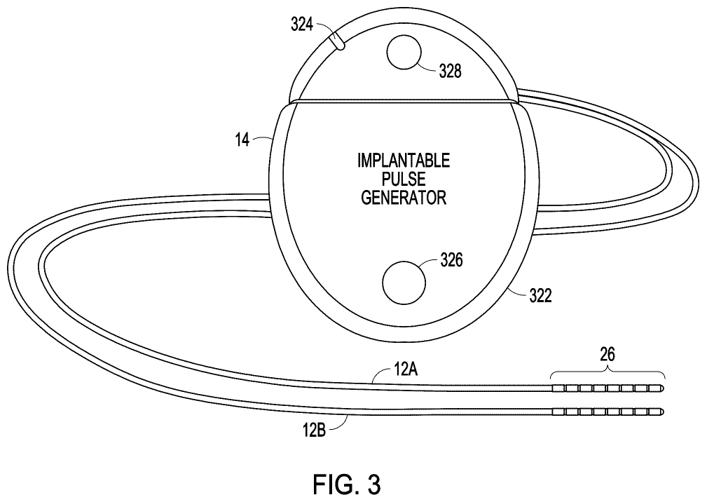

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically-sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

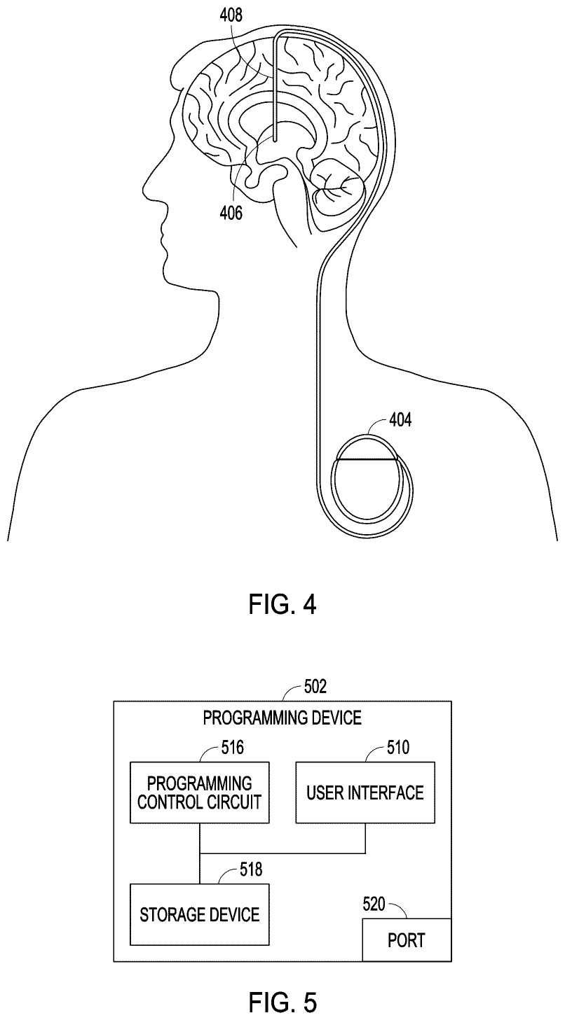

FIG. 4 illustrates an embodiment of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 5 is an illustration of an example of a programming device 502, such as programming device 102 of neurostimulation system 100 in FIG. 1. Programming device 502 includes a storage device 518, a programming control circuit 516, a user interface 510, and a port 520 (e.g., a communication port or Comm Port). Programming control circuit 516 may be implemented using an application-specific integrated circuit (ASIC) constructed to perform one or more particular functions or a general-purpose circuit programmed to perform such functions. A general-purpose circuit can include, among other things, a microprocessor or a portion thereof, a microcontroller or portions thereof, and a programmable logic circuit or a portion thereof. The storage device 518 may be a memory integral to the programming control circuit, or a separate memory device.

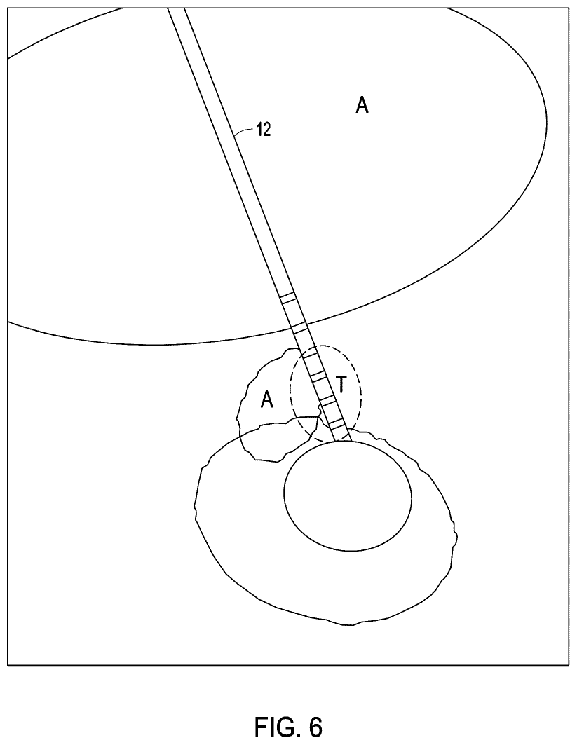

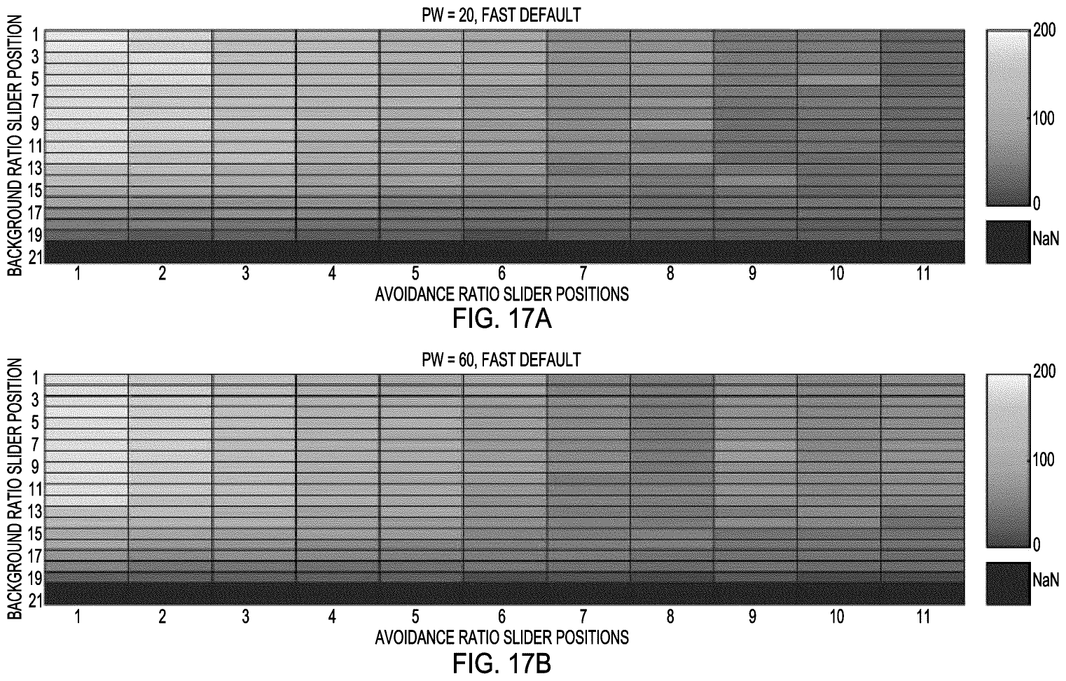

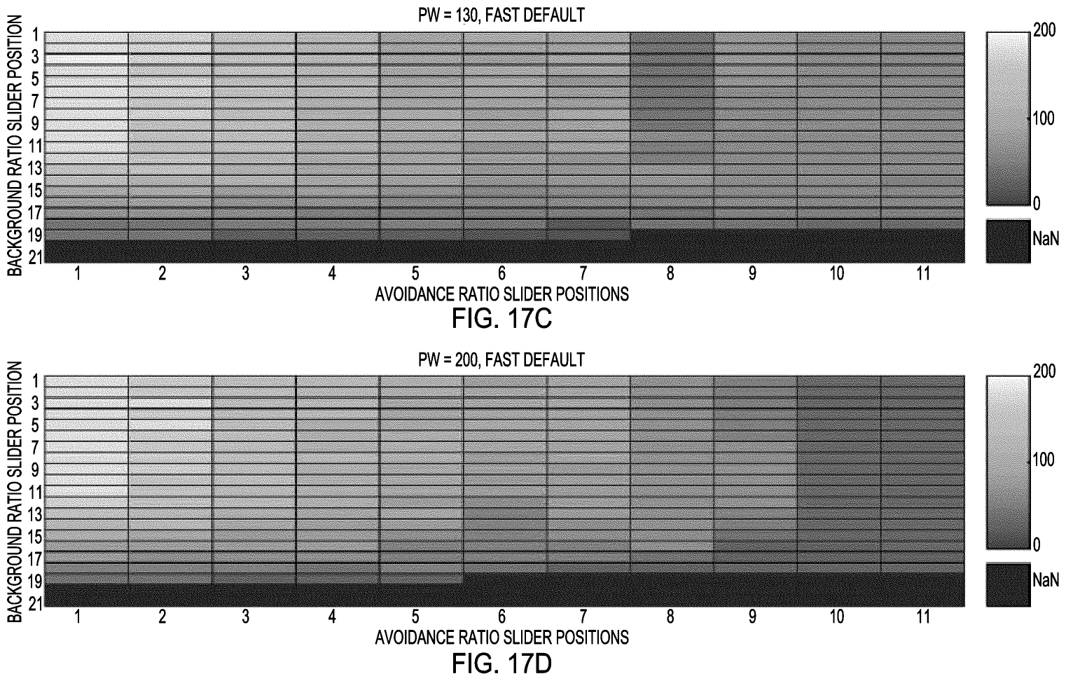

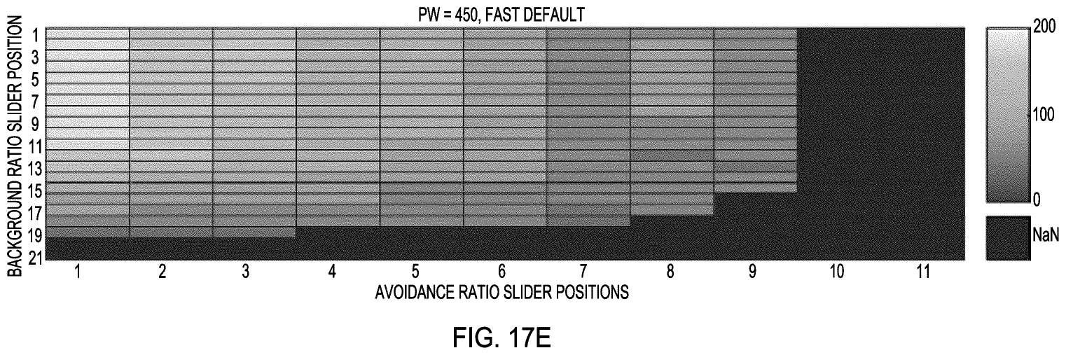

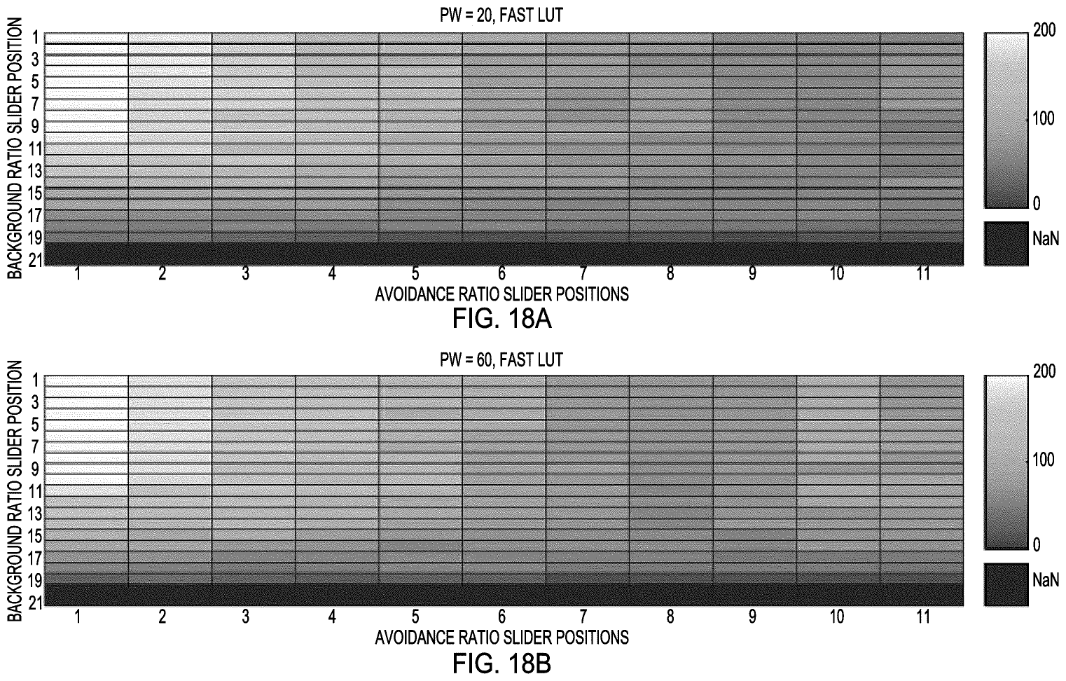

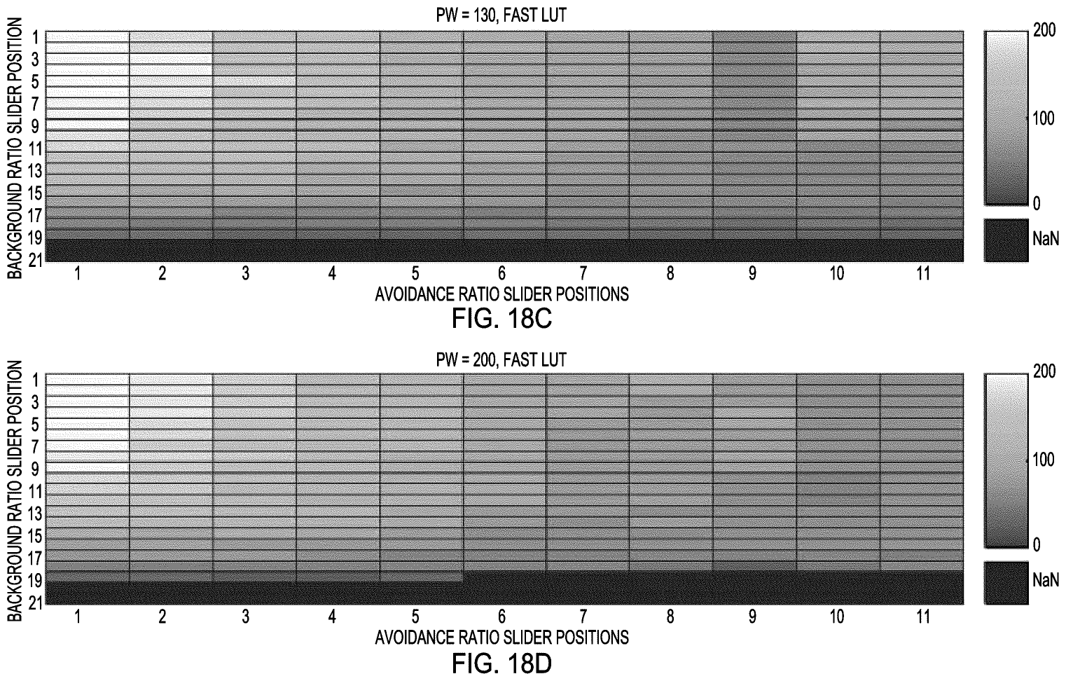

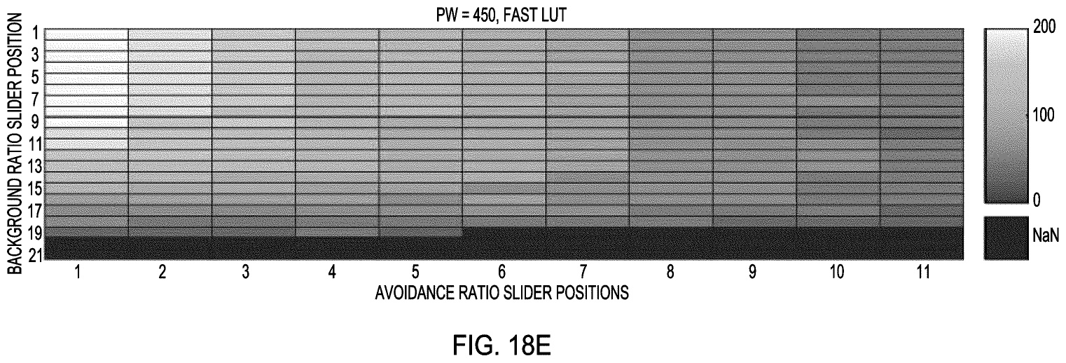

Programming control circuit 516 generates the various stimulation parameters that control the delivery of the neurostimulation pulses according to a specified stimulation configuration that can define, for example, stimulation waveform and electrode configuration. User interface 510 represents an embodiment of user interface 110 in FIG. 1. Storage device 518 stores information used by programming control circuit 516 and stimulation control circuit 320, such as information about a stimulation device (e.g., stimulation device 104 in FIG. 1) that relates the stimulation configuration to the stimulation parameters and information relating the stimulation configuration to a volume of tissue activation in the patient. In various embodiments, programming control circuit 516 can be configured to support one or more functions allowing for programming of stimulation devices, such as stimulation device 104 including its various embodiments as discussed in this document and determine a stimulation configuration solution as described in regard to FIGS. 6-18.

In various embodiments, user interface 510 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “stimulation configuration” can include the pattern of neurostimulation pulses including the one or more stimulation fields, or at least various aspects or parameters of the pattern of neurostimulation pulses including the one or more stimulation fields. Stimulation configuration can include the electrode configuration used to provide the electrical stimulation. In various embodiments, user interface 510 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

FIG. 6 is an illustration of an implanted DBS stimulation lead 12 (e.g., stimulation lead 12 of FIG. 2). The illustration in the example shows the physiological target structure (T) for the neurostimulation and avoidance structures (A) that the physician would like to avoid activating with the neurostimulation. For example, the physiological target structure may be the sensorimotor region of the subthalamic nucleus (STN) and the avoidance structures may be the nonmotor region of the STN and/or internal capsule (capsula interna).

The stimulation lead 12 includes multiple electrodes and the electrodes can be configured into multiple electrode configurations. Different electrode configurations can steer the neurostimulation energy (e.g., electrical current) toward different volumes of tissue. Additionally, the electrode configuration can include “fractionalization” of the electrical current flowing through the selected one or more electrodes. In fractionalization, a fraction of an overall pulse amplitude is assigned to each of the electrodes included in the electrode configuration.

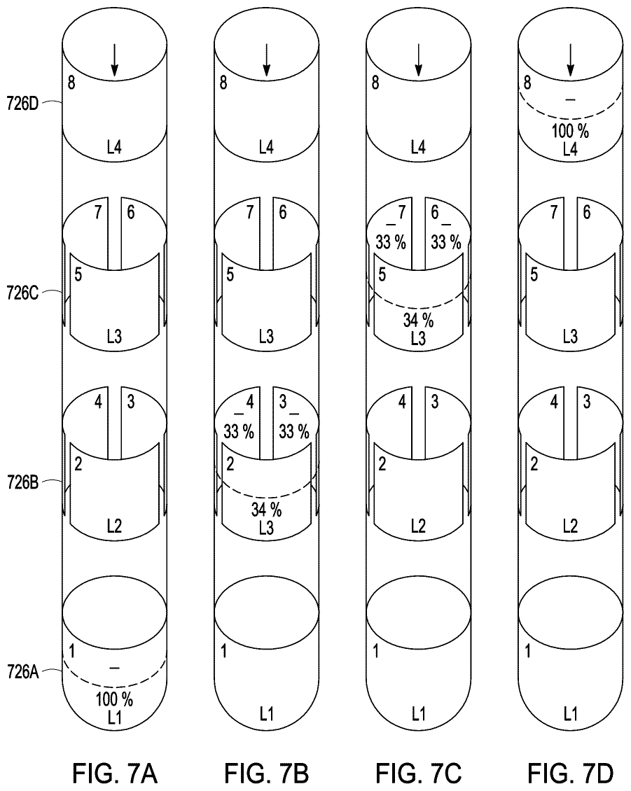

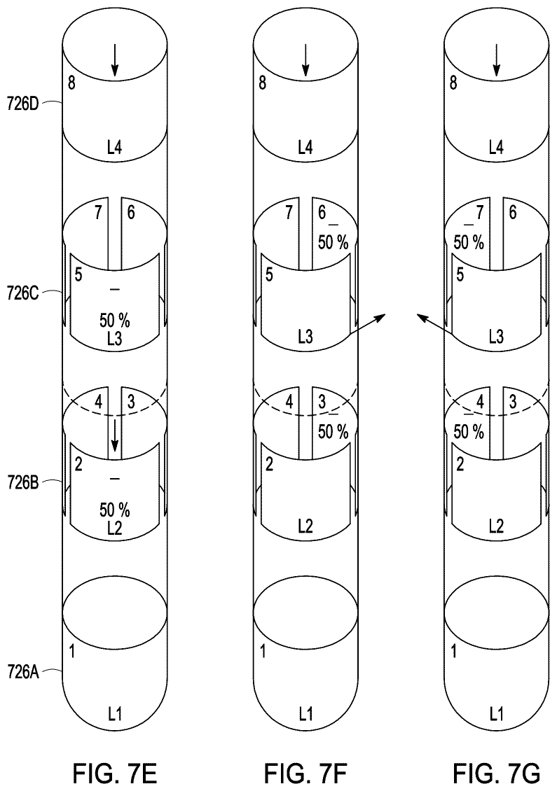

FIGS. 7A-7G are illustrations of an example of electrodes of a stimulation lead (e.g., stimulation lead 12 of FIG. 6). The electrodes are at four levels of the lead labeled L1-L4. At L1, the lead includes an electrode 726A that is a tip electrode. At levels L2 and L3, the electrodes are segmented electrodes 726B, 726C that include three electrode segments. In variations, the segmented electrodes 726B, 726C can have two segments or four segments, or segmented electrode 726B can have a different number of segments than electrode 726C. At level L4, the lead includes a ring electrode 726D. Any combination of the electrodes and electrode segments can be included in an electrode configuration, and the current delivered can be divided among the electrodes and electrode segments.

In the examples of FIGS. 7A-7D, the stimulation lead is used to provide monopolar stimulation and the electrodes that are marked with a negative sign “−” are cathodes for the stimulation. Unmarked electrodes provide no stimulation energy (e.g., current). Different electrodes are highlighted to show how the current can be delivered to different levels or heights of the electrode corresponding to different locations of the target region T is FIG. 6. Current delivered to tip electrode 726A can also emanate downward into the tissue.

In the examples of FIGS. 7E-7G, electrode configurations are shown that use segmented electrodes 726B, 726C. Segments of the electrodes are highlighted to show how current can be delivered to multiple levels and in a particular direction using electrode segments. In FIG. 7E, current is delivered to levels L2 and L4 and to the electrode segments at the front of segmented electrodes 726B, 726C. Two electrode segments are included in the electrode configuration of FIG. 7E. The current may be evenly divided among the electrode segments or different fractions of current can be provided to the electrode segments. In FIG. 7F, current is delivered to levels L2 and L4 and to the electrode segments at the right and slightly toward the rear of segmented electrodes 726B, 726C. In FIG. 7G, current is delivered to levels L2 and L4 and to the electrode segments at the left and slightly toward the rear of segmented electrodes 726B, 726C. The examples in FIGS. 7A-7G show how current can be steered by the programming control circuit 516 of FIG. 5 toward different tissue regions that are selected as the target region when the stimulation lead is implanted.

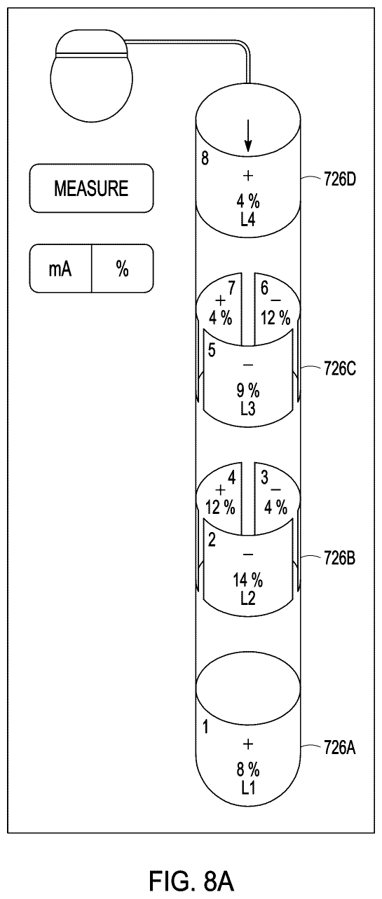

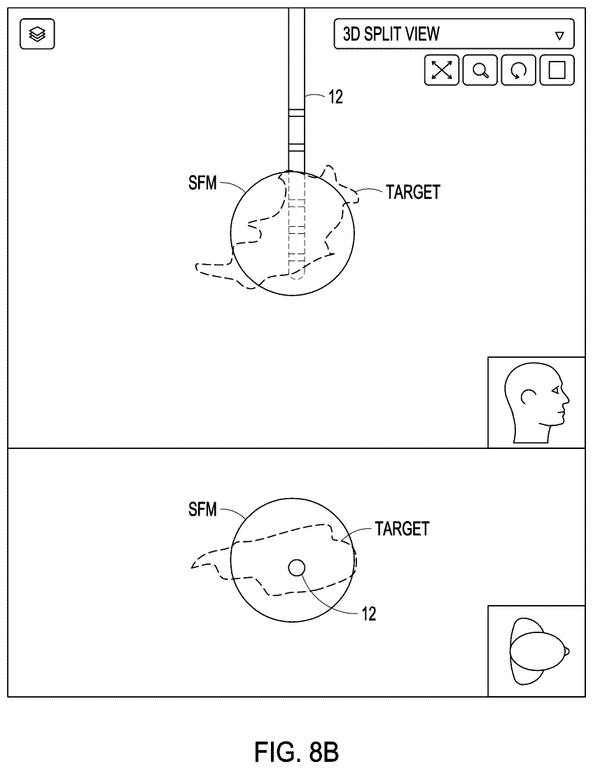

FIGS. 8A-8B illustrate an example of a presentation of a stimulation configuration using the user interface 510 of FIG. 5. FIGS. 8A-8B may be presented as one screen of a display or each Figure may be presented on a different screen. FIG. 8A presents the electrode configuration. The electrode configuration is for bipolar stimulation. In the example of FIG. 8A, the electrodes include tip electrode 726A, segmented electrodes 726C and 726B, and ring electrode 726D. The tip and ring electrodes 726A, 726D are marked with a positive sign “+” to indicate that they provide anodic current. For segmented electrodes 726B, 726C, the electrode segments marked with a “+” provide anodic current, and electrode segments marked with a “−” provide cathodic current for the stimulation. The electrodes may deliver the same fraction of the total current (i.e., 25% of the total cathodic current), or a different fractionalization can be used, varying in steps of 1% per electrode, where both anodic and cathodic stimulation sum to 100% when including the case stimulation. The stimulation on the lead may be Cathodic Major (100% of cathodic current is on the electrodes and at least some anodic current on the case), Anodic Major (100% of anodic current is on the electrodes and at least some cathodic current on the case), or Balanced, where 100% of both the Anodic and Cathodic current is on electrodes, and the case is neutral. The fractionalization of the electrode configuration may be included in the display.

FIG. 8B presents stimulation lead 12 and the target structure. The stimulation field model (SFM) is also presented. The SFM represents the total volume of tissue activated with the stimulation configuration programmed in the stimulation device (e.g., stimulation device 104 in FIG. 1). The SFM can also be referred to as a background volume. A right-side view and a top view of the stimulation lead 12, the target structure, and the SFM are presented. The display of example 8B shows the user how well the stimulation configuration covers the target structure and the extent of the tissue beyond the target structure that is affected by the neurostimulation.

As DBS systems become more complex, the number of programmable options available can become large. Neurostimulation systems can be programmable in stimulation sites, stimulation electrode combinations, stimulation pulse amplitude, pulse width, pulse rate, and pulse pattern to provide many different neurostimulation waveforms. The display of FIGS. 8A and 8B can be used as a starting point to search the parameter space to find the stimulation configuration solution with the stimulation settings that optimize the stimulation therapy for the patient. But programming to find the best combination of stimulation parameters for DBS can still be slow and labor intensive. This is becoming especially true as new systems are developed that include capabilities that expand the number of electrodes available for stimulation, provide directional stimulation steering, and provide wider stimulation parameter ranges.

To help the user find the stimulation configuration solution, the programming control circuit may perform an algorithm that includes testing to automatically find the stimulation configuration solution for the patient. There can be different approaches to designing an algorithm that automatically determines optimized neurostimulation therapy for a patient. This is especially true even when a similar optimization cost function (e.g., the metric function described later herein) is used with various mathematical techniques for optimization. Different algorithms may come up with different solutions, and there is a possibility that an algorithm may not discover the best solution available to the patient.

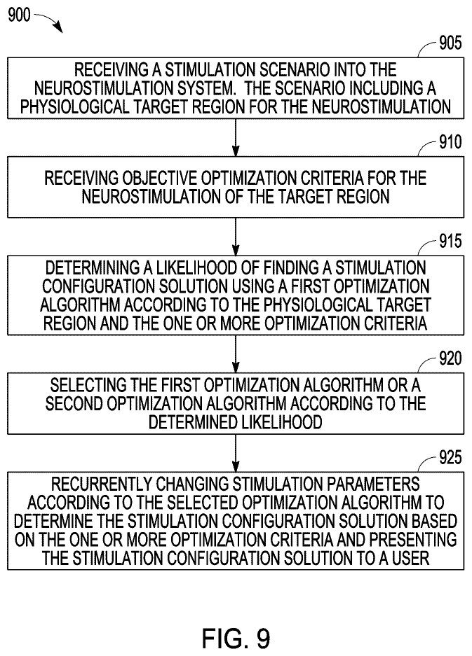

FIG. 9 is a flow diagram of a method 900 to operate a neurostimulation system (e.g., the neurostimulation system 100 of FIG. 1) to automatically determine a stimulation configuration solution for the neurostimulation system. The programming device (e.g., programming device 102 of FIG. 1, or programming device 502 of FIG. 5) of the neurostimulation system is able to perform multiple optimization algorithms. The multiple optimization algorithms are designed to automatically select a stimulation configuration solution from the stimulation settings space of the neurostimulation system. At least one of the optimization algorithms is an approximation algorithm that is designed to reduce time in identifying a stimulation configuration solution. Another optimization algorithm is more of a “brute force” algorithm. The brute force algorithm tests a set of configurations that sample on a grid in the current steering space that is available for the patient's lead type, finding the configuration that produces the optimal calculated metric for that subset of steering configurations.

At block 905, a stimulation scenario is input to the neurostimulation system. The input stimulation scenario provides information on placement of the stimulation lead or leads with respect to one or more structures of the patient's brain. The stimulation scenario may designate the target stimulation structure (e.g., a motor STN or the LSCC) that is the target region for the stimulation. The target region may be input to the system by the user via the user interface (e.g., user interface 110 of the programming device 102 in FIG. 1), or the target region may be input to the system from a separate device communicatively coupled to a port of the programming device (e.g., port 520 of the programming device 502 of FIG. 5). Some examples of the separate device include a magnetic resonance spectroscopy system or a tomography system.

The input stimulation scenario may also designate any avoidance structures or avoidance regions that the stimulation should avoid activating. The input stimulation scenario can include information regarding the energy of the neurostimulation, such as values for the pulse width (PW) of the stimulation.

At block 910, optimization criteria are input into the neurostimulation system. One or more optimization criteria can be input by the user via the user interface. Examples of the optimization criteria include the upper limit for the stimulation pulse amplitude and/or the total charge to be delivered to with each stimulation pulse. Additional examples of optimization criteria include the fill volume of and the spill volume. The fill volume includes the absolute or percent volume of tissue within the target region that is stimulated, and overlaps the SFM. Spill volume refers to the volume of tissue included in the SFM but outside the target region. Another example of an optimization criterion can be a ratio including the fill volume and spill volume.

Other examples of optimization criteria include the minimum clinical effects response to the stimulation. The clinical effects response may specify a therapeutic benefit of the stimulation (e.g., reduction of tremors) and avoidance of side effects of the stimulation such as rigidity, muscle contractions, psychiatric side effects, etc. Options for the optimization criteria can be presented using menus of a GUI.

At block 915 in FIG. 9, the programming device of the system decides which of the optimization algorithms to use in determining the stimulation configuration solution. The algorithms use one or more decision criteria to select a stimulation configuration. One example of a decision criterion is an optimization metric. The metric includes a cost function of the fractionalization of the stimulation configuration. The metric is a weighted summation of the tissue volumes stimulated by the neurostimulation energy. The tissue volumes include the target region or target volume (v_{target}), any avoidance volumes (v_{avoidance}), and the background volume (v_{SFM}). In the weighting, the target volume is given a positive weight, and the avoidance volumes and the background volume are given negative weights.

The negative weights can be modified by cost or weighting factors. For example, the negative weight of the avoidance volumes can be modified by an avoidance ratio such as the ratio of the cost of stimulating the avoidance volume to the benefit of stimulating the target volume. The negative weight of the background volume can be the ratio the cost of stimulating a unit volume of the background tissue to the benefit of stimulating a unit volume of the target tissue.

The equation for the value of the metric (m) is:

$$m = \sum ( {v}_{target} - ( {v}_{a v o i d a n c e} * avoidance ratio ) - ( {v}_{SFM} * background ratio ) ) .$$

The volumes may be expressed in cubic millimeters (mm³). The avoidance ratio and the background ratio may be input as part of the stimulation scenario or the optimization criteria for the scenario, which can be manually selected by the user or selection of the ratios may be guided by a user interface. The neurostimulation system can include a GUI and the user can select the avoidance and background ratios using the GUI (e.g., selecting the ratios using sliders of the GUI). Stimulations configurations with a higher value of the metric provide more neurostimulation benefit to the patient.

Using the selected optimization algorithm, the programming device tests stimulation configurations identified by the algorithm by configuring the stimulation device with candidate test configurations, applying the neurostimulation to the patient, and evaluating the results of the candidates according to the objective optimization criteria.

As explained previously herein, some optimization algorithms may be designed to test a large subset or nearly every stimulation configuration that the neurostimulation can provide to determine the exact best stimulation configuration solution. For example, the brute force algorithm always tests the parameter search space, or a specified subset of the parameter search space, the same way—by testing all combinations of the parameter search space.

Some optimization algorithms are designed to conclude faster with a recommended simulation configuration that, while befitting the patient, may not be the best possible stimulation configuration (i.e., the stimulation configuration solution) that the neurostimulation system can provide for the particular patient. For example, a faster optimization algorithm may stop testing stimulation configurations as soon as a stimulation configuration with a target value for the metric m is found or if a specified limit on the number of optimization steps is achieved.

In another example, a faster algorithm may initially sample a small number of settings around an initial estimate. The initial small number of settings may be determined according to a heuristic. The faster algorithm may use a gradient descent approach to reach a conclusion faster. The algorithm may construct an approximation of the metric surface based on those settings, and the approximate surface is used to determine a new setting to test, giving another metric value. This provides more data to refine the approximation of the metric surface, which it then uses to determine another setting to test. The algorithm continues this process until certain criteria are met (e.g., a maximum number of tests has been conducted, or the new settings stop giving better solutions). Once the criteria are met, the algorithm returns the stimulation settings giving the best results. These stimulation settings may correspond to a local optimum of the metric surface rather than the global optimum although the goal is to find the global optimum. The algorithm is faster than the brute force type algorithm because it tests fewer settings to reach a conclusion.

The programming device may determine the likelihood that one or more of the faster optimization algorithms will find the stimulation configuration solution. The programming device/software may further determine the likelihood that one of the optimization algorithms will find the “better” stimulation configuration solution. The programming device may use the input stimulation scenario to determine which optimization algorithm will find the better solution. The programming device may perform an analysis of initial conditions such as the position of the lead with respect to the target, the shape of the target region, and the position of the lead with respect to the avoidance regions to determine whether the faster algorithm will find the better stimulation configuration solution than another algorithm. The programming device may further perform an analysis of the optimization criteria set by the user to determine the likelihood that the faster algorithm will find the better stimulation configuration solution. In another example, machine learning can be used to implement a model trained to predict the algorithm performance based on the characteristics of the input scenario and/or the optimization criteria.

Based on the determined likelihood of finding a stimulation configuration solution and that the solution is the better solution for the patient, the programming device may select either a faster optimization algorithm or a slower more thorough algorithm at block 920. If the likelihood is high that a faster optimization algorithm will find the better stimulation configuration solution, the programming device selects the faster optimization algorithm. For example, the programming device may select the faster optimization algorithm when determining that the probability of that algorithm finding the better stimulation configuration solution is greater than a predetermined probability threshold.

At block 925, the programming device tests stimulation configurations according to the selected optimization algorithm. The programming device recurrently changes stimulation parameters of the stimulation device delivering the neurostimulation. The stimulation configuration that meets the optimization criteria, or best meets the criteria, is presented to the user as the stimulation configuration solution. The GUI may present an option to select the presented solution or allow the user to make changes to one or more parameters of the presented solution and then selecting the resulting stimulation configuration.

According to some examples, the programming device may search a database to determine the likelihood of finding a stimulation configuration solution using a particular optimization algorithm. The database may be stored in a storage device of the system (e.g., in a memory of the programming device) or the database may be stored in a cloud server. The database may store stimulation configuration solutions determined for a patient population using that particular optimization algorithm. The database stores the objective requirements for the stimulation configuration solutions. The database may be organized as a look up table. Finding stimulation configuration solutions in the database that have similar requirements for the current stimulation configuration search increases the likelihood that the optimization algorithm will find a stimulation configuration solution for the current search.

In some examples, the programming device searches the database for a match in the target region. Finding a match in the target region means that a stimulation configuration solution for that target region was previously found by the optimization algorithm, and the probability increases that the optimization algorithm will find a solution for the present search. The programming device may look for additional matching criteria. Matching additional information increases the probability of that the optimization algorithm will find a solution for the present search. In some examples, the programing device searches for a match in both the target region and the avoidance regions. In some examples, the programing device searches for a match in electrode configuration. In some examples, the programing device searches for a match in one or more of the optimization criteria.

Based on the extent of the match or matches of stored configurations to the present search, the programming device may seed the search using stimulation parameters of the matched configurations. For example, the programming device may configure the stimulation device with a candidate electrode configuration that is based on a matched configuration. This may shorten the time needed by the optimization algorithm to find an optimized current steering method for a stimulation configuration solution in the present search.

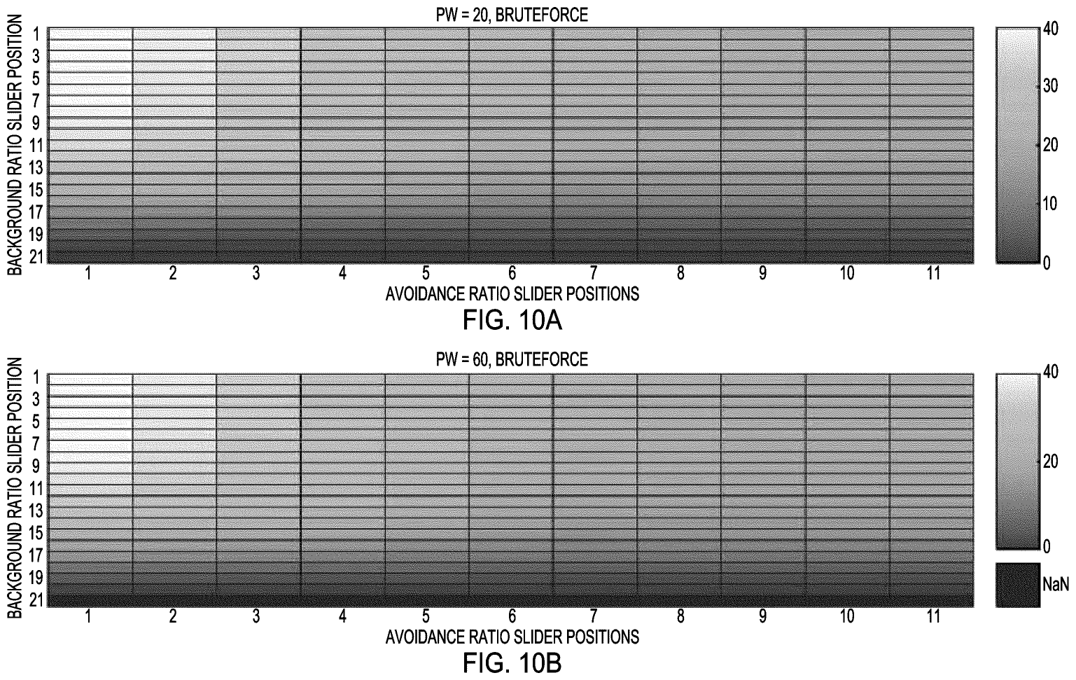

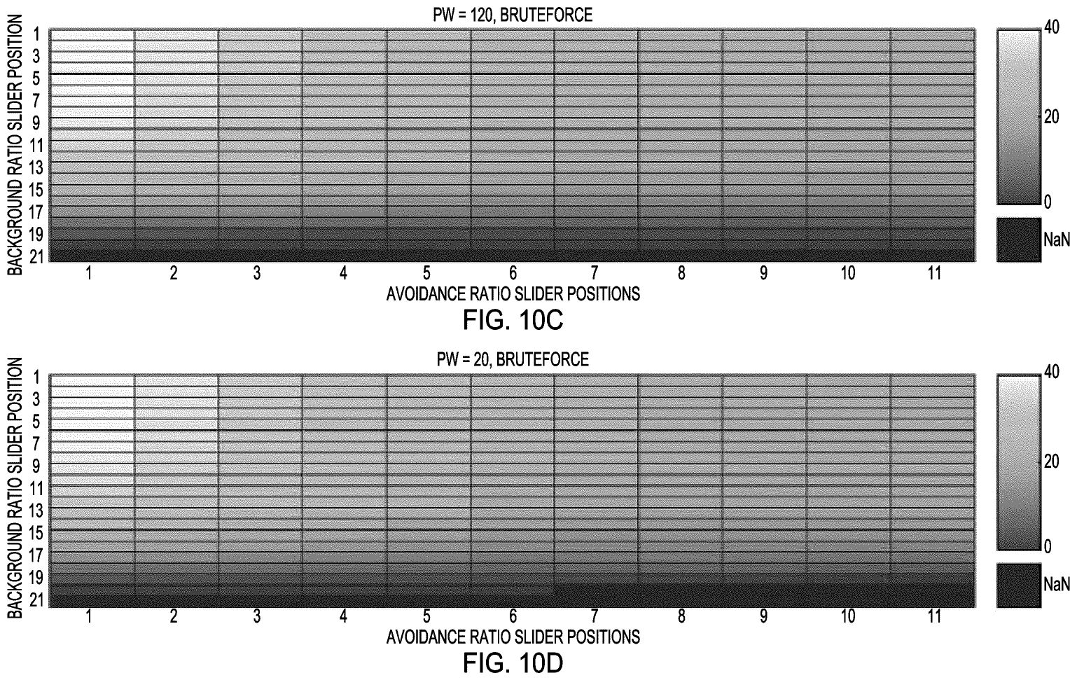

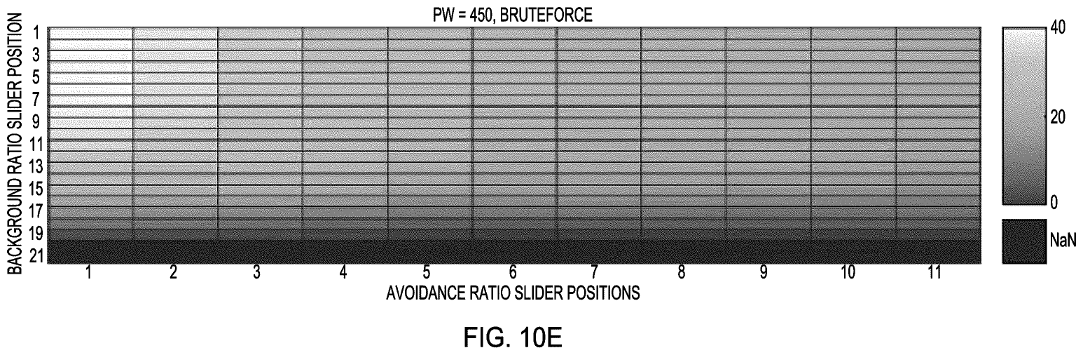

FIGS. 10A-10E are graphs of results of finding a stimulation configuration solution using a brute force type optimization algorithm. The optimization criterion is the value of the metric (m). The horizontal axis of each graph is 11 different values or positions of the avoidance ratio slider of the GUI. The horizontal axis is 21 different positions of the background ratio slider of the GUI. Note that value of the background ratio is increasing in the downward direction. Thus, each graph comprises 231 results—one result for each combination of avoidance ratio and slider ratio. Each result in the graphs is an optimized result for the corresponding values of avoidance ratio and background ratio. Each graph shows the results for a different pulse width (PW) in milliseconds (ms). The five values of PW in the test examples are 20 ms, 60 ms, 130 ms, 200 ms, and 450 ms. Changing the position of the slider changes the values of the ratios in the equation for the metric (m) described previously herein.

The key shown to the right of the graphs shows the value of the metric obtained. The upper left area in the graphs is the area where the cost of stimulating background and avoidance regions is the least, so the SFM can be the largest, resulting in stimulating more target volume, and resulting in a higher values of the metric. The results of the upper rows have a lower cost of stimulating background. The stimulation amplitude can be larger and stimulates more of the target, so the metric is higher. The leftmost columns have a lower cost of stimulating the avoidance regions, also allowing the stimulation amplitude to be higher and stimulating more of the target region. However, stimulating a larger volume increases the chance of a side effect occurring.

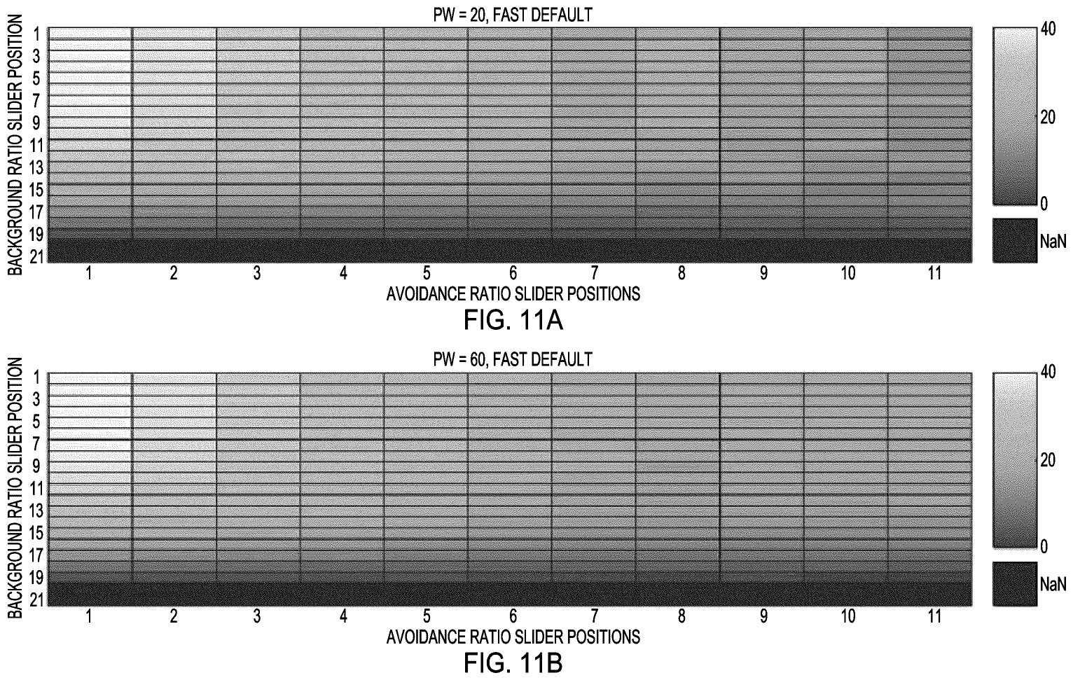

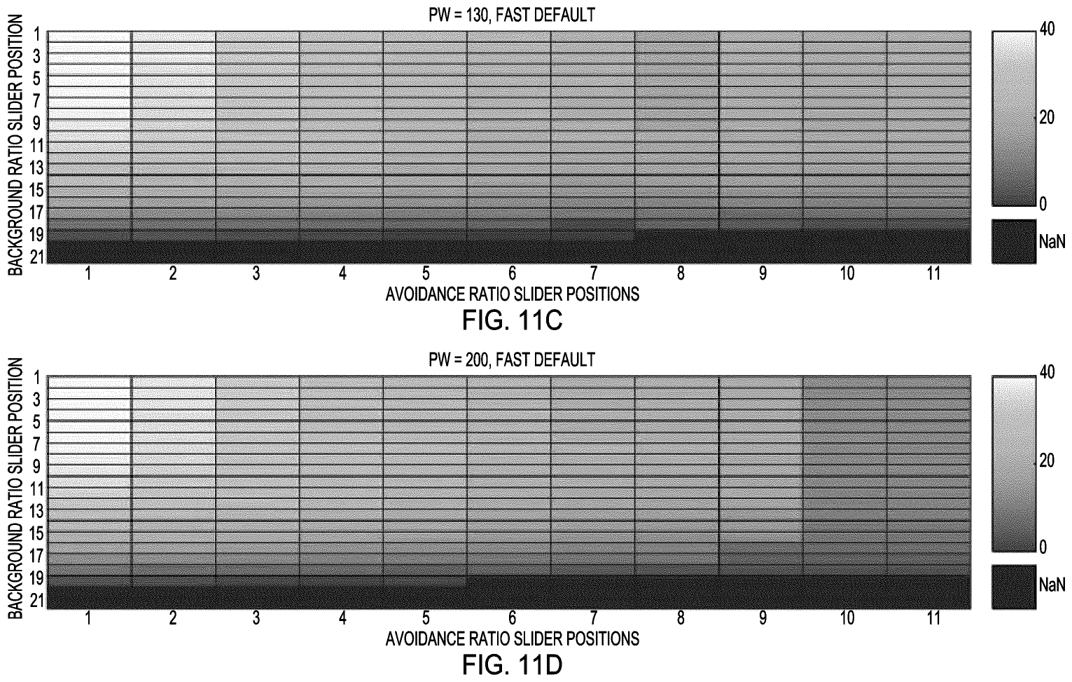

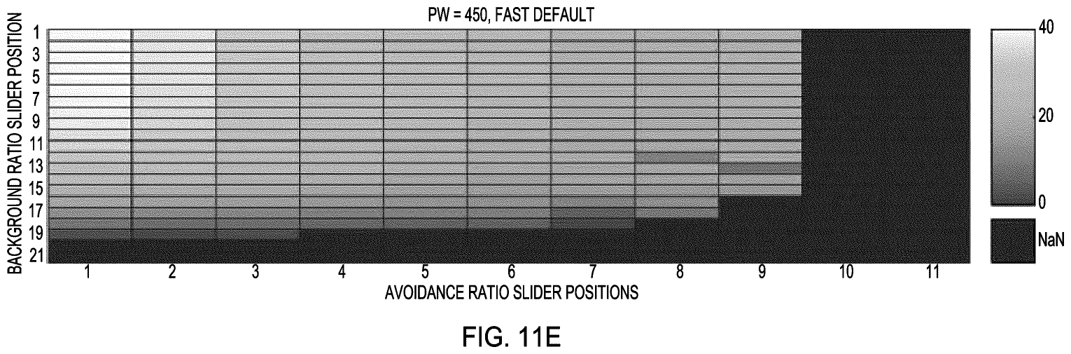

FIGS. 11A-11E shows graphs results of finding a stimulation configuration solution using a faster optimization algorithm (Fast Default). The faster algorithm uses a gradient descent approach that samples the metric value at certain places in the steering space and constructs a representative metric surface. It uses the constructed surface to determine a new position to sample and updates the surface. This process continues until certain stop criteria are met and the best stimulation configuration is returned. Various optimization algorithms are available to search the steering space.

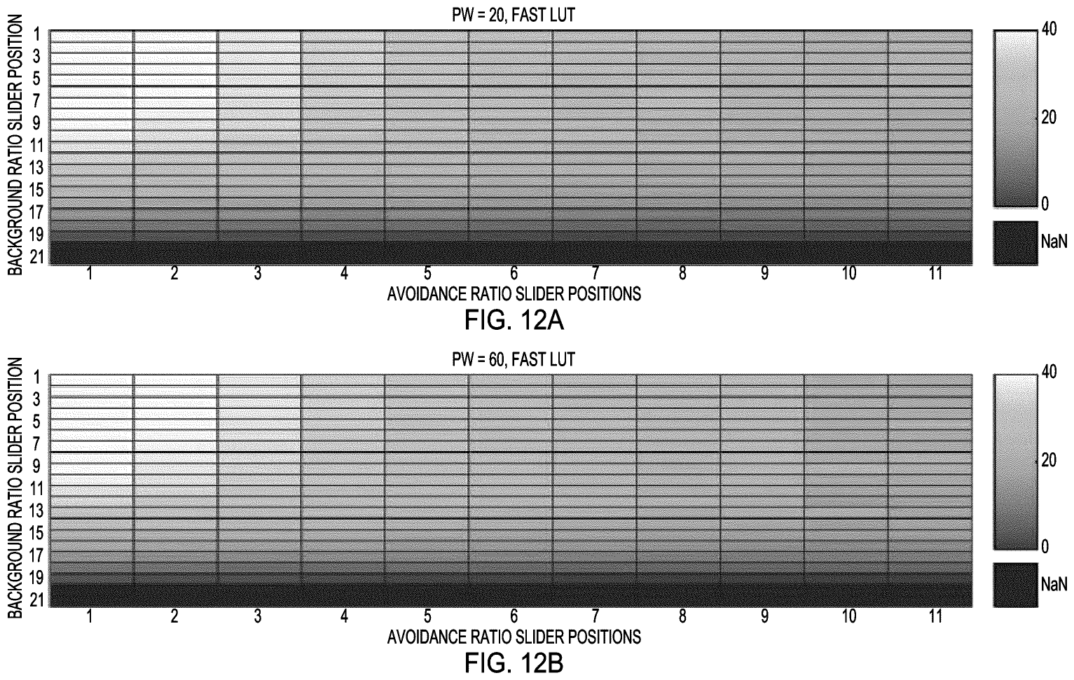

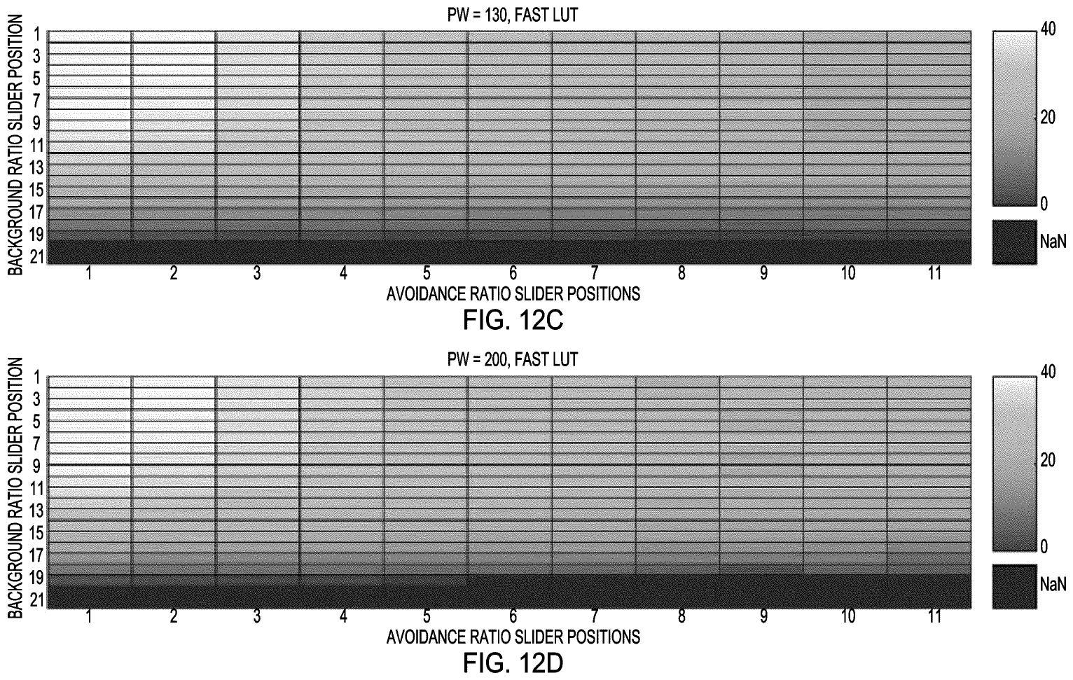

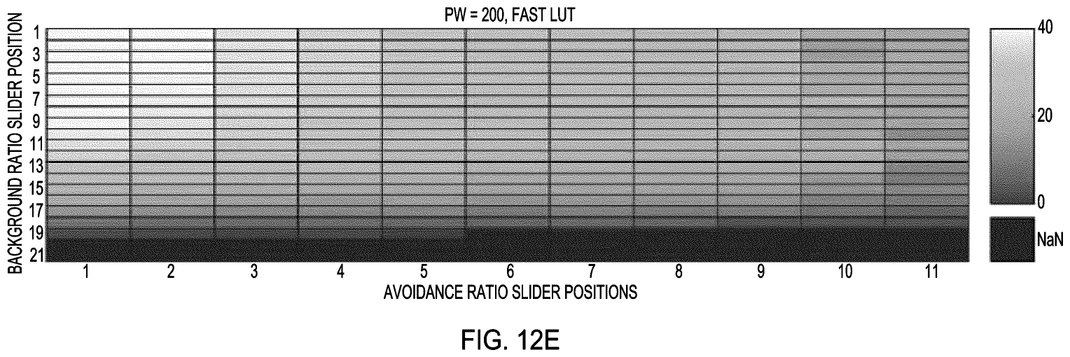

The optimization algorithm of FIGS. 11A-11E uses the default electrode configurations for current steering to search the search space for a solution. FIGS. 12A-12E are graphs of results of finding a stimulation configuration solution using the faster optimization algorithm but with database searching or database lookup (Fast LUT). The programming device searches the database to find candidate electrode configurations for current steering. The graphs show that the faster optimization algorithm with database lookup performed better than the faster optimization algorithm with default steering at higher values of background and avoidance ratios.

Based on the results in FIGS. 10A-12E, the programming device may select the faster optimization algorithm approach of FIGS. 11A-11E when the optimization criteria include the lower values of background and avoidance ratios, and the objective is to maximize the metric (m). When the background and avoidance ratios are lower, there is a higher likelihood that the faster algorithm will find the best the stimulation configuration solution in less time than the brute force algorithm will find the stimulation configuration solution. Similarly, the programming device may select the faster optimization algorithm approach of FIGS. 12A-12E when the optimization criteria include somewhat higher values of background and avoidance ratios. The faster algorithms typically take a few seconds to complete while the brute force algorithm may take minutes to complete. However, the programming device may determine that the brute force algorithm is likely to find a better stimulation configuration for the patient than what will be found by the faster algorithms. The programming device may select the slower brute force approach of FIGS. 10A-10E when the optimization criteria include the highest values of background and avoidance ratios and the likelihood of finding the stimulation configuration solution using one of the faster optimization algorithms is lower.

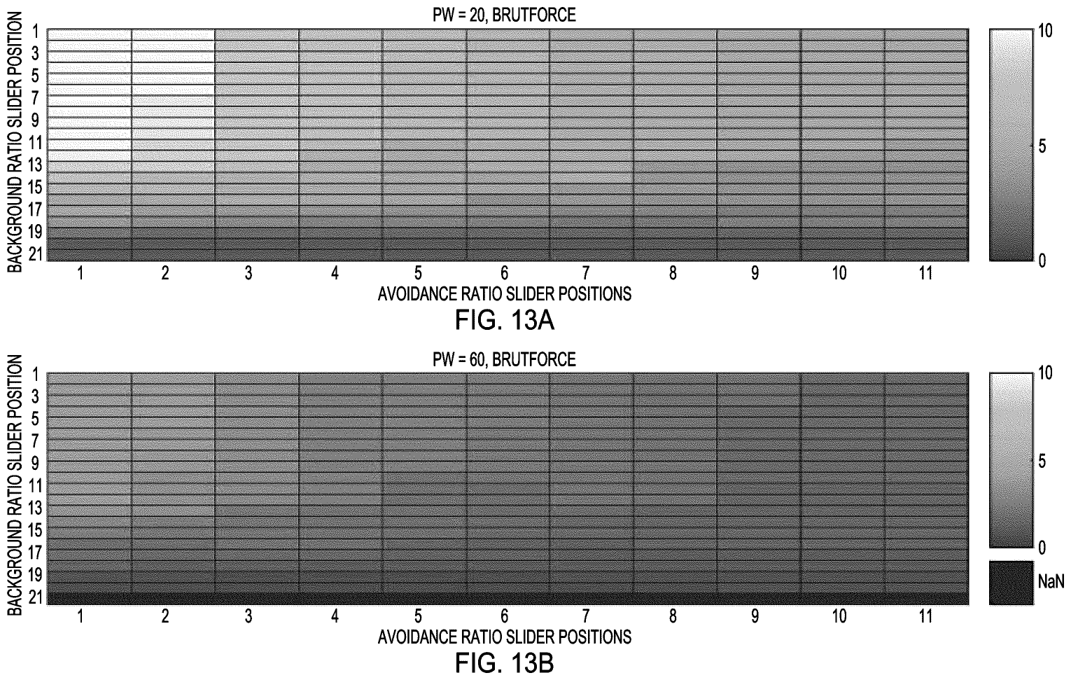

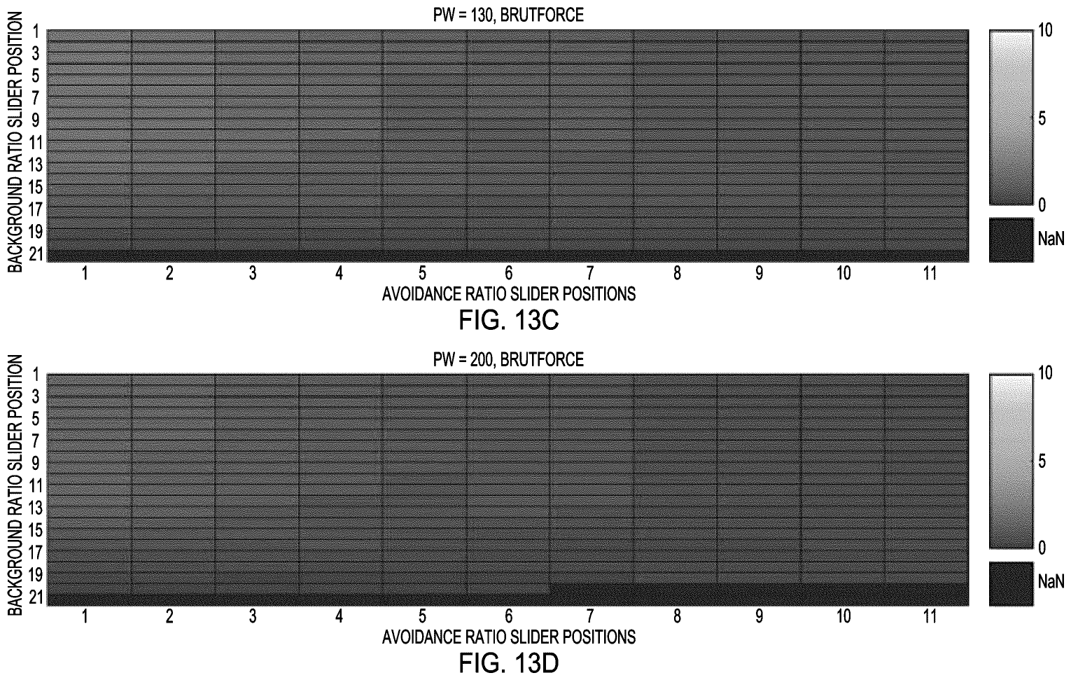

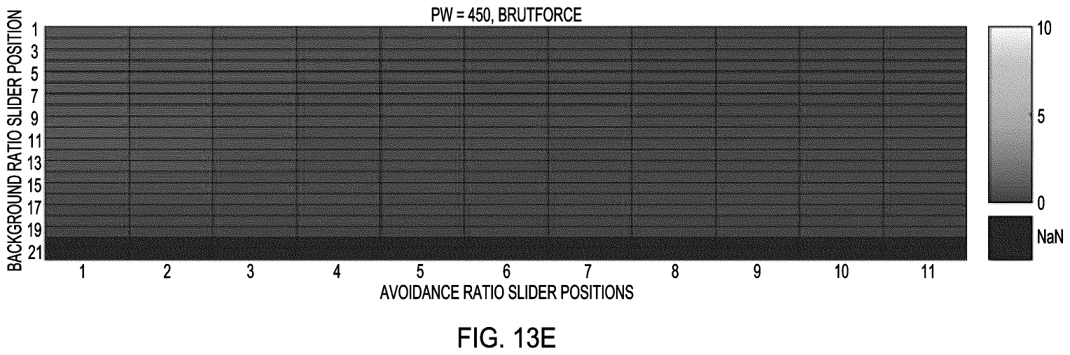

FIGS. 13A-15E are show graphs of results of finding a stimulation configuration solution. The optimization objective is the value of the stimulation amplitude. FIGS. 13A-13E, 14A-14E, 15A-15E correspond to using the brute force type optimization algorithm, the faster optimization algorithm with default steering methods, and the faster optimization algorithm with look up searching for steering methods, respectively. The pulse width (PW) is varied as in the examples of FIGS. 10A-12E with the objective of finding the solution stimulation amplitude that adequately activates the target region. The stimulation amplitude can be an amplitude, or it can be specified as a radius of the stimulation from the stimulation lead. The key shown to the right of the graphs show that optimization of the stimulation amplitude is given a scale of 0-10 with 10 being the optimized amplitude.

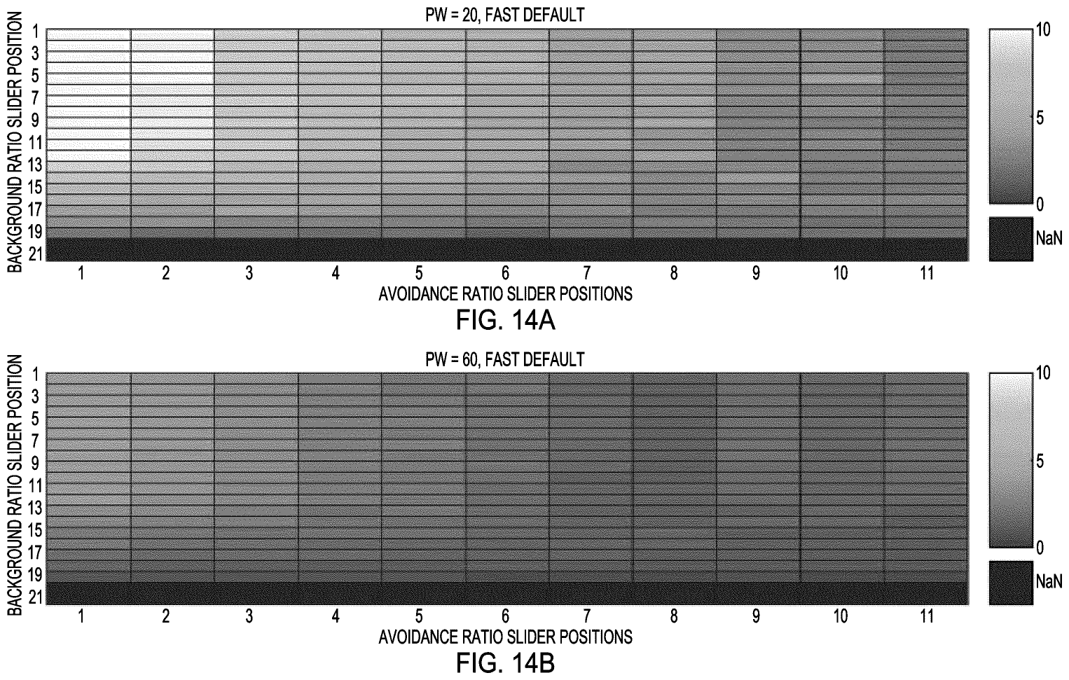

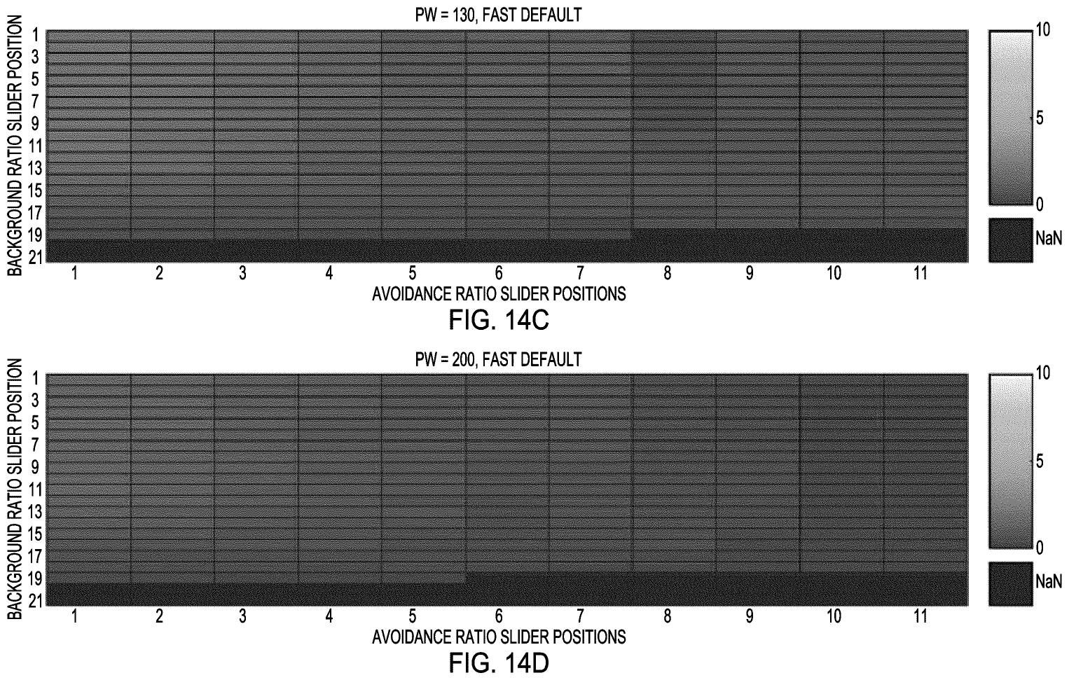

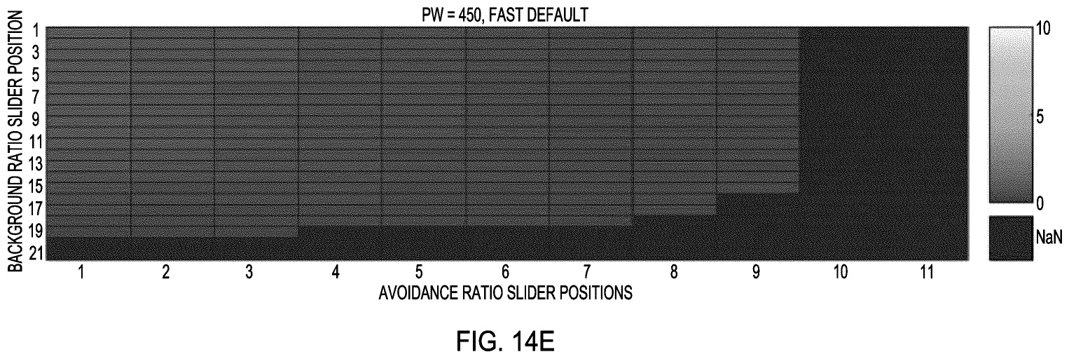

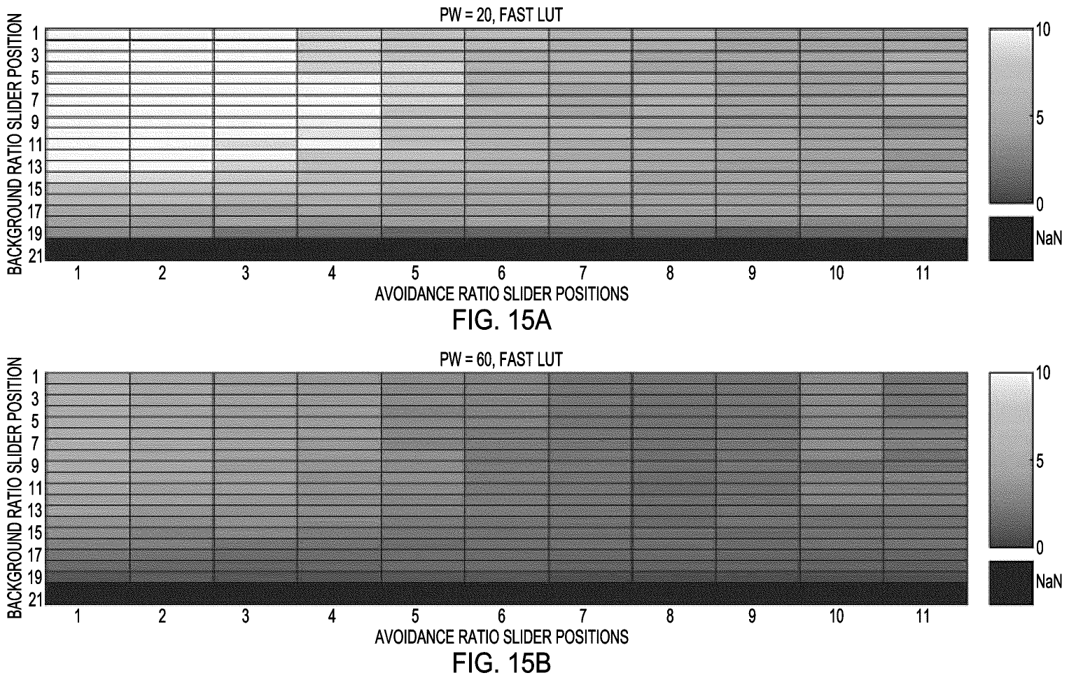

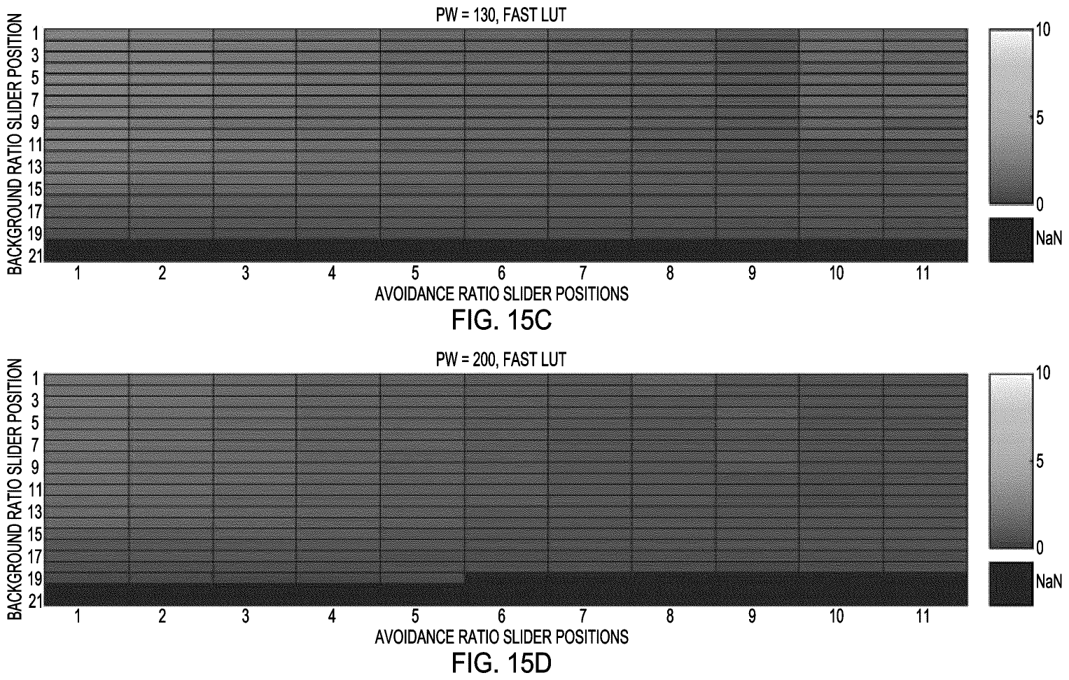

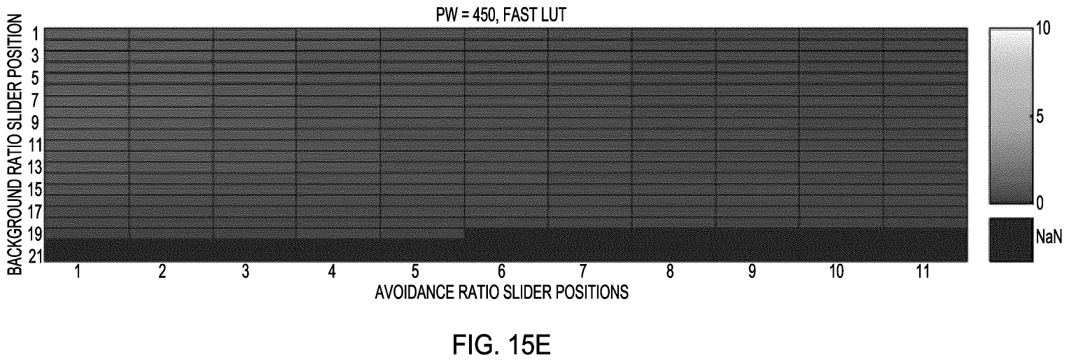

A comparison of FIGS. 13A-13E and 14A-14E show that the faster optimization algorithm with default steering did not perform as well as the brute force method at the higher background and avoidance ratios. But a comparison of FIGS. 13A-13E and 15A-15E show that the faster optimization algorithm with look up searching performs better than the brute force approach for some midrange values of the background and avoidance ratios. The programming device may select one of the faster optimization algorithms based on the likelihood of the algorithm finding a stimulation configuration solution for selected values of the background and avoidance ratios and the likelihood that the identified stimulation configuration solution is a better solution for the patient than what would be found by the other algorithms.

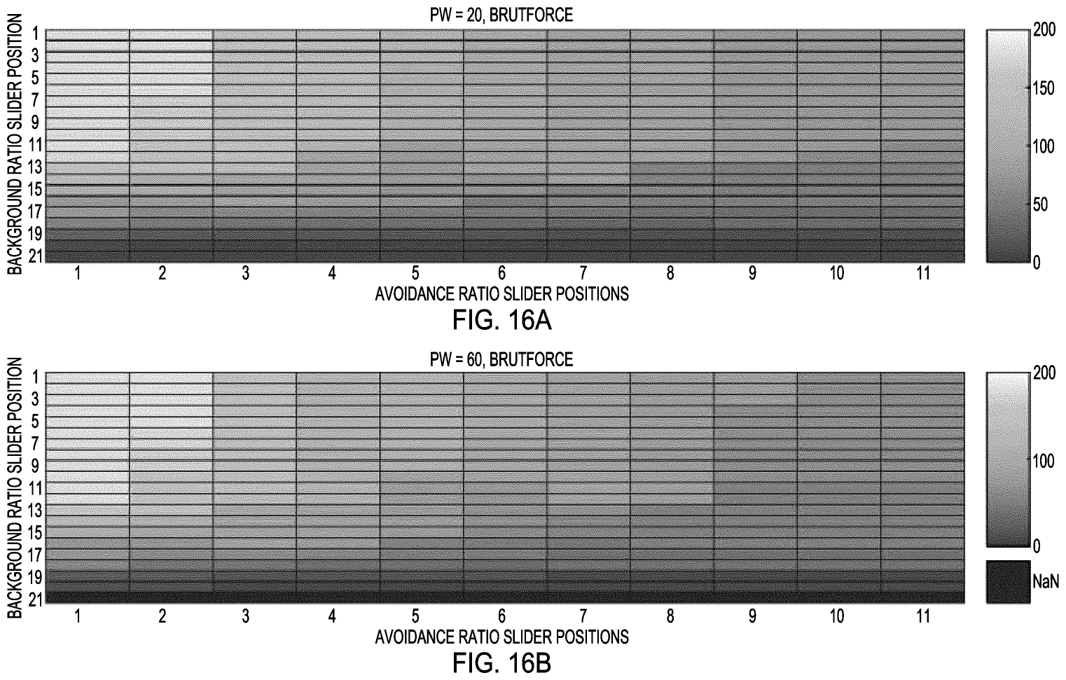

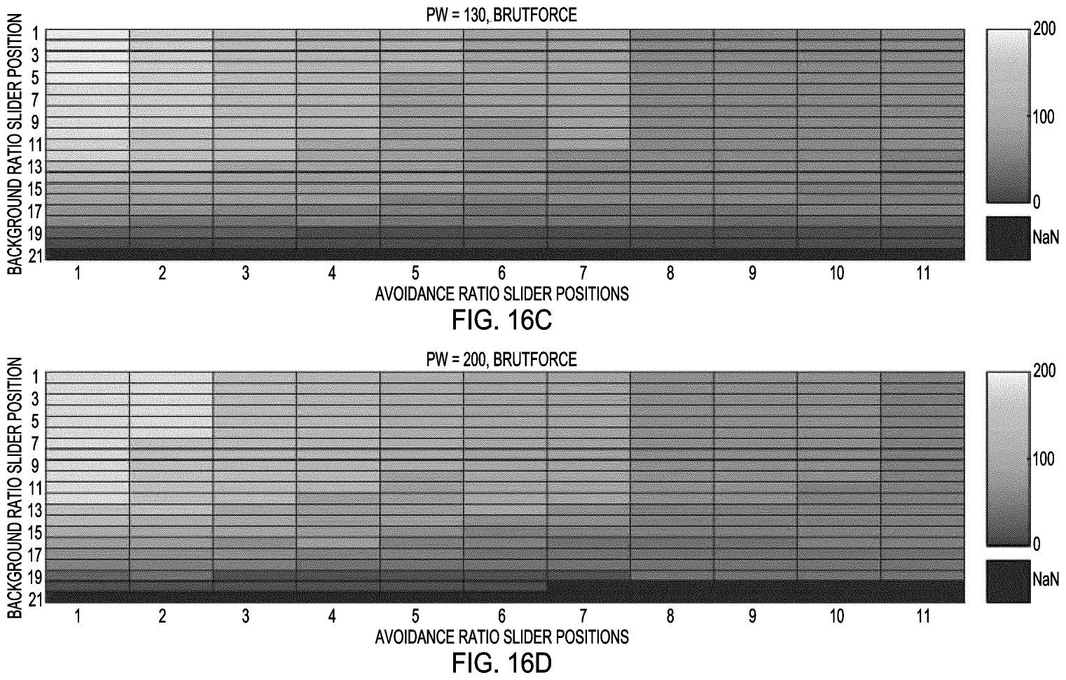

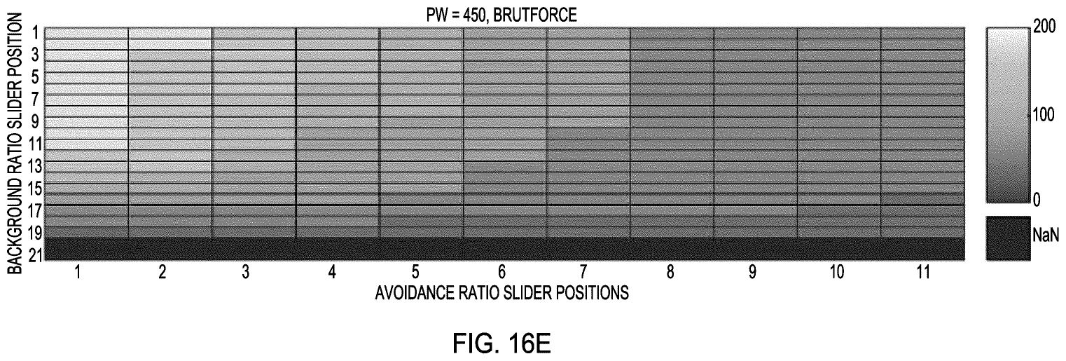

FIGS. 16A-18E each show graphs of further examples of results of finding a stimulation configuration solution. The optimization objective specified by the user is the SFM volume. FIGS. 16A-16E, 17A-17E, and 18A-18E correspond to using the brute force type optimization algorithm, the faster optimization algorithm with default steering methods, and the faster optimization algorithm with look up searching for steering methods, respectively. The pulse width is varied as in the examples of FIGS. 10A-12E with the optimization objective of optimizing the SFM to meet the target stimulation while avoiding undesirable side effects. The programming device may select one of the faster optimization algorithms based on the likelihood of the algorithm finding a stimulation configuration solution that meets the objective for the SFM volume. Additional examples of optimization objectives include the ratio of the fill volume to spill volume the clinical effects from the stimulation.

The devices, systems and methods described herein provide techniques for neuroanatomy-based searching to find optimized deep brain stimulation therapy for a particular patient quickly. These techniques will continue to be useful as the search space for the optimized stimulation continues to grow with advances in DBS therapy.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A method of controlling operation of a neurostimulation system to deliver neurostimulation to tissue of a patient using a plurality of electrodes, the method comprising:
receiving, by the neurostimulation system, a target region for the neurostimulation, wherein the target region is physiological;
receiving one or more optimization criteria for the neurostimulation of the target region;
determining a first likelihood of finding a stimulation configuration solution using a first optimization algorithm according to the target region and the one or more optimization criteria;
determining a second likelihood that the stimulation configuration solution found by the first optimization algorithm is a better stimulation configuration solution than a stimulation configuration solution that would be found by a second optimization algorithm;
selecting the first optimization algorithm or a second optimization algorithm according to the determined first and second likelihoods; and
recurrently changing stimulation parameters according to the selected optimization algorithm to determine the stimulation configuration solution based on the one or more optimization criteria and presenting the stimulation configuration solution to a user.

2. The method of claim 1,
wherein finding the stimulation configuration solution includes finding a solution electrode configuration, the electrode configuration specifying a selection of one or more electrodes from the plurality of electrodes and a fractionalization of electrical current flowing through the selected one or more electrodes;
wherein the first optimization algorithm uses a gradient descent approach to find the solution electrode configuration; and
wherein the second optimization algorithm tests every available electrode configuration of the neurostimulation system to find the stimulation configuration solution.

3. The method of claim 1, including:
receiving, by the neurostimulation system, one or more avoidance regions for the neurostimulation; and
wherein determining the stimulation configuration solution includes:
determining a weighted summation including a stimulated target volume of the target region, a stimulated avoidance volume of the one or more avoidance regions, and a total stimulated volume; and
determining the stimulation configuration solution according to the weighted summation.

4. The method of claim 1, wherein the determining the first likelihood of finding the stimulation configuration solution and the second likelihood that the stimulation configuration solution is a better stimulation configuration solution includes:
searching a database of stored stimulation setting results of a patient population, the stored stimulation setting results determined using the first optimization algorithm; and
determining the first and second likelihoods according to matching the received target region to target regions for the stored stimulation setting results of the database.

5. The method of claim 4, wherein the determining the first likelihood includes determining the likelihood of finding the stimulation configuration solution according to matching one or more electrode configurations of the neurostimulation system to electrode configurations for the stored stimulation setting results of the database.

6. The method of claim 4, wherein recurrently changing stimulation parameters includes determining, when the first optimization algorithm is selected, test stimulation configurations using stimulation parameters of matched stored stimulation setting results of the database.

7. The method of claim 1, wherein the optimization criteria include a ratio including the total tissue volume activated by the neurostimulation and the tissue volume outside of the target region activated by the neurostimulation.

8. The method of claim 1, wherein the optimization criteria include optimization of one or both of stimulation amplitude and total charge delivered to the tissue of the patient.

9. The method of claim 1, including:
receiving, by the neurostimulation system, one or more avoidance regions for the neurostimulation; and
wherein at least one of the first or second optimization algorithm identifies a simulation configuration solution using a decision criterion that includes a value of a metric that includes a weighted summation of a stimulated target volume of the target region, a stimulated avoidance volume of the one or more avoidance regions, and a total stimulated volume of the tissue of the patient.

10. A system for delivering neurostimulation to tissue of a patient using multiple electrodes, the system comprising:
a stimulation control circuit to deliver the neurostimulation according to a specified stimulation configuration, the stimulation configuration including multiple stimulation parameters;
a port to receive a designation of a target region for the neurostimulation;
a user interface configured to receive one or more optimization criteria for the neurostimulation of the target region; and
a programming control circuit configured to:
specify multiple stimulation configurations;
perform multiple optimization algorithms, each algorithm to determine a stimulation configuration solution from the multiple stimulation configurations;
determine a first likelihood of finding a stimulation configuration solution using a first optimization algorithm according to the target region and the one or more optimization criteria;
determine a second likelihood that the stimulation configuration solution found by the first optimization algorithm is a better stimulation configuration solution than a stimulation configuration solution that would be found by a second optimization algorithm;
select the first optimization algorithm or another optimization algorithm according to the determined first and second likelihoods; and
recurrently change stimulation parameters according to the selected optimization algorithm to determine the stimulation configuration solution based on the one or more optimization criteria and present the stimulation configuration solution using the user interface.

11. The system of claim 10, wherein the programming control circuit is configured to:
specify electrode configurations that include a selection of one or more electrodes of the multiple electrodes;
identify, when performing the first optimization algorithm, an initial set of stimulation settings, find an approximate stimulation configuration solution using the initial set of stimulation settings, and identify a next set of stimulation settings based on the approximate stimulation configuration solution; and
when performing the other optimization algorithm, test every available electrode configuration of the multiple electrodes when finding the stimulation configuration solution.

12. The system of claim 10, wherein the programming control circuit is configured to:
receive one or more avoidance regions for the neurostimulation;
determine the stimulation configuration solution using a weighted summation including a stimulated target volume of the target region, a stimulated avoidance volume of the one or more avoidance regions, and a total stimulated volume; and
determine the stimulation configuration solution according to the weighted summation.

13. The system of claim 10, including:
a storage device to store a database of stimulation setting results determined for a patient population using the first optimization algorithm, and
wherein the programming control circuit is configured to:
search the database to identify stored stimulation setting results for the received target region; and
determine the first likelihood of finding the stimulation configuration solution according to the identified stored stimulation setting results.

14. The system of claim 13, wherein the programming control circuit is configured to:
select one or more electrode configurations of the multiple electrodes to activate the target region; and
determine the first likelihood of finding the stimulation configuration solution and determine the second likelihood that the stimulation configuration solution is the better stimulation configuration according to matching the one or more electrode configurations of the neurostimulation system to electrode configurations for the stored stimulation setting results of the database.

15. The system of claim 14, wherein the programming control circuit is configured to recurrently change stimulation parameters to stimulation settings of matched stored stimulation setting results of the database when using the first optimization algorithm to determine the stimulation configuration solution.

16. The system of claim 13, wherein the programming control circuit is configured to:
search the database to identify stored stimulation setting results that include at least one optimization criterion of the received one or more optimization criteria; and
determine the first likelihood of finding the stimulation configuration solution according to the stored stimulation setting results identified according to the at least one optimization criterion.

17. The system of claim 16, wherein the at least one optimization criterion includes a weighting of activation of tissue outside the target region.

18. A non-transitory computer readable storage medium including instructions that when performed by a programming control circuit of a neurostimulation device, cause the neurostimulation device to perform actions including:
receiving, by the neurostimulation device, a target region of a subject for neurostimulation, wherein the target region is physiological;
receiving one or more optimization criteria for the neurostimulation of the target region;
determining a first likelihood of finding a stimulation configuration solution using a first optimization algorithm according to the target region and the one or more optimization criteria;
determining a second likelihood that the stimulation configuration solution found by the first optimization algorithm is a better stimulation configuration solution than a stimulation configuration that would be found by a second optimization algorithm;
selecting the first optimization algorithm or a second optimization algorithm according to the determined first and second likelihoods; and
recurrently changing stimulation parameters according to the selected optimization algorithm to determine the stimulation configuration solution based on the one or more optimization criteria and presenting the stimulation configuration solution to a user.

19. The non-transitory computer readable storage medium of claim 18, including instructions that cause the neurostimulation device to:
search a database of stored stimulation setting results of a patient population stored in a storage device, the stored stimulation setting results determined using the first optimization algorithm; and
determine the first likelihood of finding the stimulation configuration solution according to matching the received target region to target regions for the stored stimulation setting results of the database.

20. The non-transitory computer readable storage medium of claim 18, including instructions that cause the neurostimulation device to:
identify, when performing the first optimization algorithm, an initial electrode configuration, wherein an electrode configuration specifies a selection of one or more electrodes from the plurality of electrodes and a fractionalization of electrical current flowing through the selected one or more electrodes;
find an approximate electrode configuration solution using the initial electrode configuration and identify a next electrode configuration based on the approximate electrode configuration solution;
select a solution electrode configuration from the candidate electrode configurations; and
test, when performing the second optimization algorithm, every electrode configuration available to the neurostimulation system to determine the solution electrode configuration.
